(19) United States
(12) Patent Application Publication
(10) Pub. No.: US 2024/0412859 A1
(43) Pub. Date: Dec. 12, 2024

(54) DEVICE, SYSTEM AND METHOD FOR PROCESSING PATIENT SENSOR DATA

(71) Applicant: QUANTIUM MEDICAL SLU, Mataro (ES)

(72) Inventors: UMBERTO MELIA, Sant Pol Demar (ES); Erik Weber Jensen, Sant Pol Demar (ES); Gabriel Garcia Hernando, Barcelona (ES); Carmen Gonzalez Pijuan, Barcelona (ES); Joana Canellas Santos, Tarragona (ES); Pol Castane Guzman, Arenys de Mar (ES)

(21) Appl. No.: 18/699,268

(22) PCT Filed: Oct. 7, 2022
(86) PCT No.: PCT/EP2022/077967

§ 371 Date: Apr. 6, 2024

(30) Foreign Application Priority Data
Oct. 7, 2021 (EP) 21382899.9

(51) Int. Cl. G16H 40/63; A61B 5/00; G06F 21/62

(52) U.S. Cl. CPC G16H 40/63; A61B 5/7475; G06F 21/6245

(57) ABSTRACT

An electronic device (1) for processing patient (P) sensor data comprises one or more sensing devices (10) for measuring a physical parameter with respect to the patient's (P) body; and a control unit (11) adapted to: provide, via one or more communication interfaces (110) of the control unit (11), authentication data for authenticating the electronic device (1) at a monitoring device (20); acquire sensor data indicative for measurement values of the physical parameter with respect to the patient's (P) body using the one or more sensing devices (10); and provide, via the one or more communication interfaces (110), the sensor data.

The invention relates to a device, to a system and to a method for processing patient sensor data.

Monitoring a patient is desirable in various situations related to medical treatments/operation, like during intensive care, relocation of patients especially during administering a drug to a patient. For example, when administering a drug to the patient, particularly an anaesthetic drug, or during a target-controlled infusion (TCI). TCI generally refers to an infusion operation performed by a computer-assisted infusion system which calculates a substance concentration in a particular body compartment on the basis of a mathematical model and which, after setting a target concentration, adjusts the infusion rate such that the concentration in the body compartment of the patient converges towards and is kept at the predefined target concentration. TCI infusion systems generally consist of one or multiple infusion devices and a control device, which may be separate to the infusion devices or may be integrated into an infusion device.

For setting up an infusion operation, herein, patient specific parameters such as the patient's age, weight, gender, and drug-specific parameters such as the type of drug, e.g. the type of anaesthetic, and a desired target concentration in a body compartment of the patient, for example relating to a drug level in the patient's brain within an anaesthesia procedure, may be entered into the system using a human-machine interface.

On the basis of an empirically determined population-pharmacokinetic model and using a known pharmacokinetic and patient-specific pharmacodynamic parameter set of a medicament (for example propofol) as well as by means of patient-specific data, a TCI system may model a drug distribution (over time) within the patient's body by calculating drug concentrations in body compartments as defined within the model. During the execution of a target-controlled infusion protocol, herein, the mathematical model may be repeatedly adjusted according to measurement values of one or more physical parameters with respect to the patient's body, e.g., relating to a drug concentration within the patient, for example by measuring a drug concentration in a patient's breath or in the patient's plasma (blood) compartment, or by measuring (electro-) physiological data, e.g., biological signals such as EEG or ECG signals, or by deriving indices such as the so-called bi-spectral (BIS) index. According to the measurement values the mathematical model can be used during operation such that it suitably reflects the concentrations in the patient's body compartments, such that patient-individual effects, e.g., a patient-specific metabolism, may be taken into account. The measurement of the one or more physical parameters thus allows to obtain a desired medical effect, such as an anaesthetic effect during an anaesthesia procedure.

To obtain the desired medial effect, and, also, to have knowledge of the patient's status precisely, it is desirable to obtain reliable measurement values of one or more physical parameters with respect to the patient's body.

It is an object of the instant invention to acquire and provide sensor data indicative for reliable measurement values of one or more physical parameters with respect to a patient.

This object is achieved by means of an electronic device, by means of a system and by means of a method comprising the features of the respective independent claim.

According to an aspect, an electronic device for use in a medical operation is provided. The term medical operation in this case means any medical treatment of action taken in a medical environment. For example, but not limited to intensive care, relocation of patients and especially during administering a drug to a patient. The electronic device comprises one or more sensing device(s) (each) for measuring a physical parameter with respect to the patient's body, and a control unit. The control unit is adapted to provide, via one or more communication interfaces thereof, authentication data for authenticating the electronic device at a monitoring device external to the electronic device, to acquire sensor data indicative for measurement values of the physical parameter with respect to the patient's body using the one or more sensing devices, and to provide, via the one or more communication interfaces, the acquired sensor data. Hence, the control unit is adapted to enable a communicative connection between the monitoring device and the electronic device. Furthermore, the control unit comprises at least one memory.

The control unit may be directly integrated into the sensing device. Alternatively, the control unit may be a separate part comprised in the electronic device.

For example, the control unit may be a dedicated chip mounted on or in the sensing device.

Hence, the electronic device may be a smart sensor, and it allows to determine, before using the electronic device to record sensor data of the electronic device, whether or not the electronic device is suitable for the respective operation. For example, the electronic device may be unsuitable if it does not provide a functionality and/or measurement precision needed for the operation, if it has been used for too long, if it has been used for another patient before, or the like. This allows to provide more reliable measurement values of one or more physical parameters with respect to a patient. The authentication data may be unique for the given electronic device and/or for its type among a plurality of different types of electronic devices. The different types of electronic devices may comprise sensing devices that are adapted for measuring different physical parameters, and/or physical parameters at different locations of the patient's body. The one or more communication interfaces can be embodied in hardware and/or software. The physical parameter with respect to the patient's body may be, e.g., an (electro-) physiological parameter.

According to one embodiment the electronic device comprises sensing elements which are EEG electrodes. The electronic device contains a flexible strip, containing a flexible circuit, with three or more EEG electrodes arranged thereon to collect EEG signals. Furthermore, a secure element is contained in the control unit, also attached to the flexible strip. In use the flexible strip is arranged along the forehead to one temple of the patient. The fixed positions of the electrodes along the strip ensure proper placement of the sensing elements on the patient's forehead and temple.

For example, the electronic device communicatively connects the sensing devices and the control unit with the monitoring device by means of a cable. Other wireless connections are also possible, for example Bluetooth, infrared, RFID, Wi-Fi or the like.

The electronic device, in particular the control unit of the electronic device, may comprise a secure element adapted to provide the authentication data. The secure element may be adapted to provide access to secured information, e.g., the authentication data or other stored data, only under specific preconditions. For example, the secure element provides access to secured information only if a predefined cryptographic key and/or password and/or other predefined information is provided to the secure element. This allows to secure sensitive information and a reliable and secure operation. The secure element may be a dedicated chip mounted on or in the control unit. Alternatively, the secure element may be part of a system-on-a-chip (SoC) of the control unit.

Optionally, the electronic device, particularly the secure element of the electronic device, comprises a cryptographic key (either encrypted or not) and/or a cryptographic algorithm. This may be supplied with, or as, or to generate the authentication data. This allows a particularly secure communication.

The authentication data may consist of, or comprise, an encrypted cryptographic key, or, more generally, an encrypted piece of information. This may be decrypted by the monitoring device with a certain key. For example, the monitoring device may send the key or piece of information to the electronic device. The electronic device then encrypts it with the cryptographic algorithm, and provides it with, or as, the authentication data. The cryptographic algorithm may be a symmetric cryptographic algorithm based on a shared key (shared by the monitoring device and the electronic device), or it may be an asymmetric cryptographic algorithm based on one or more private and public key pairs. For example, the control unit (particularly the secure element thereof) of the electronic device can store the public key of the monitoring device, and/or vice versa. This allows to ensure that the electronic device is only used with a (e.g., specific) monitoring device in order to avoid combinations with a lower reliability and/or with a precision lower than necessary for a specific operation, or other unsuitable combinations.

Alternatively, or in addition, the authentication data may be indicative for the type of the electronic device and/or for the type of a suitable monitoring device. This also allows to avoid combinations with a lower reliability and/or with a precision lower than necessary for a specific operation, or other unsuitable combinations.

These embodiments ensure that an advice on drug dosage, for example an anaesthetic dosing can only be provided to a user as long as specific electronic device is used, which is identified by its secure element and as long as adequate conditions (e.g. not expired) are met.

Especially in an advisory system (for example an open loop system) this behaviour provides an additional security layer, meaning that the electronic device will only be able to provide data to the monitoring device in case the secure element is present and properly pre-programmed.

The control unit comprises at least one memory, in particular non-volatile memory, or a combination of volatile and non-volatile memory for storing data. For example, memory of the control unit may be part of the secure element. Alternatively, or in addition, memory of the control unit may be external of the secure element. Optionally, such memory external to the secure element may be accessible by the secure element (optionally accessible only by the secure element). Memory allows to store specific data as described in more detail below, and by this, a particularly reliable function.

The memory stores expiry data to indicate and/or calculate an expiry date of the electronic device. The control unit is adapted to provide the stored expiry data via the one or more communication interfaces, e.g., to the monitoring device that is in communicative connection with the electronic device. This allows to avoid the use of an electronic device after the end of its lifetime and, thus, to increase the reliability of the measurement. The expiry data may for example include the manufacture date of the electronic device and a usage time. Alternatively, or in addition, the expiry data can comprise a specific expiry date. In general, the expiry data allows the monitoring device to determine whether the electronic device has expired and needs a replacement or can be used. For example, in case the monitoring device determines that the expiry data has passed or that a usage time has exceeded, e.g. determining invalidity, a warning message is generated, that indicates to a user that the sensor has expiry. The warning message may be for example an acoustic signal and/or a visual signal, or the like generate by the monitoring device. Furthermore, the screen of the monitoring device might freeze, disabling further usage of the electronic device.

In addition, the control unit may be adapted to store sensor data and/or anaesthesia data and/or patient data in the memory, in particular all of this data. The anaesthesia data may be indicative for a type of anaesthesia. The patient data may be indicative for the name, age, weight, gender and/or health condition or the like. By this, the electronic device may be continuously or repeatedly used with the same patient, but with different monitoring devices. After the data has been stored in the memory, a further use of the electronic device with the same patient and another monitoring device may be simplified and more reliable. For example, the electronic device may store data received from a monitoring device in the memory and provide it (particularly after successful authentication) to another monitoring device.

For example, in case that a patient is transferred from one hospital unit to a different one, for example from an intensive care unit (ICU) to an operating room (OR) and within the ICU and the operating room different monitoring device are used, data already stored in the memory of the electronic device can be of use, in particular if the monitoring device in the ICU is a device for monitoring the depth of anaesthesia.

Therefore, the electronic device could be able to provide a signal to enable potential different type of monitoring algorithmics depending on the different characteristics of the patient such as age, health condition, etc. For example, paediatric and geriatric general anaesthesia could significantly differ from adults in the brain's activity response.

In addition, or alternatively, the control unit may be adapted to store usage data in the memory. Meaning, that the control unit stores information about in which operation and/or for how long the electronic device has already been used. These information may be used to determine an enhanced expiry date, which expires before the initially stored expiry date. For example, if the electronic device has been used for a long period of time on the verge of the limit, this might lead to an earlier expiry date than it has initially been stored to the memory. Also using the electronic device multiple times for only short periods of time may result to a reduced usage time and thus, an earlier expiry date. By this an additional insurance relating to the reliability of measurement values of one or more physical parameters with respect to the patient's body is given. In addition, or alternatively the expiry date is set as production date+x months. If a usage is detected, usage data are generated and the enhanced expiry date is set 24 hours after the first use of the electronic device. In addition, or alternatively, the expiry date, or the enhanced expiry date will be reached if the sensing device or the sensing devices are removed for three periods of time, which lasted longer than a predetermined period of time. The predetermined period of time is preferably in the range of a couple of minutes, because the conductive properties of the electrodes of the sensing device(s) worse with each removal.

In other words, the monitoring device determines whether the electronic device has expired by calculation of time used and/or disconnections or by comparison of the stored expiry date vs the date of the monitoring device.

The control unit may be adapted to receive data (e.g., some or all of the data described above) via the one or more communication interfaces, e.g., from the monitoring device, and to store the received data in the memory.

Each of the one or more sensing devices may be (or comprise) an electrode, e.g., to acquire (electro-) physiological data of the patient as sensor data. This particularly allows reliable anaesthetic procedures.

According to an aspect, a system for processing patient sensor data is provided. The system comprises an electronic device, e.g., the electronic device according to any aspect or embodiment described herein. The system further comprises a monitoring device (e.g., as described above or elsewhere herein). The monitoring device comprises one or more communication interfaces and an authentication module. The one or more communication interfaces can be embodied in hardware and/or software. The one or more communication interfaces may particularly be adapted to communicate with (the) one or more communication interfaces of the electronic device. The system (e.g., the monitoring device of the system) is adapted to receive (via the one or more communication interfaces thereof), (the) authentication data from the electronic device, to determine, by means of the authentication module, the validity of the received authentication data; and to record, based on the determined validity of the received authentication data, the sensor data received from the electronic device. This allows an improved reliability of the measurement. The authentication module may be embodied in hardware and/or software. Furthermore, the monitoring device is adapted to receive the expiry data sent by the communication interface of the electronic device.

For example, the monitoring device may be adapted to disable the recording of sensor data if the received authentication is determined to be invalid. In that, the sensor data is only recorded if the authentication was successful. As an example, the electronic device may acquire sensor data and/or send sensor data independently of the authentication, and the monitoring device receives it, but only records it if the authentication was successful (i.e., the authentication data was determined to be valid). Alternatively, or in addition, upon a successful authentication, the monitoring device sends a feedback message to the electronic device, acknowledging the successful authentication. The electronic device may then (e.g., only then) acquire and/or send sensor data to the monitoring device upon reception of the feedback message.

In addition, if the electronic device is beyond its expiry date, the monitoring device may show a warning. According to one embodiment it will proceed with recording the sensor data. According to an alternative embodiment it will refuse to record the sensor data.

Additionally, the monitoring device may be adapted to check whether or not, the expiry date is reached during a current operation and if so, it will show a warning.

The system may comprise an infusion device, particularly for administering anaesthetic drugs. The infusion device may be a part of the monitoring device, or it may be separate thereof. The infusion device is in communicative connection with (e.g., the remainder of) the monitoring device. Alternatively, or in addition, the system can be adapted to determine and/or monitor a depth of anaesthesia based on the sensor data.

According to an aspect, a method for processing patient sensor data is provided. The method comprises:
1) providing, via one or more communication interfaces of a control unit of an electronic device, authentication data for authenticating the electronic device at a monitoring device;
2) providing, via one or more communication interfaces of a control unit of the electronic device, expiry data of the electronic device at the monitoring device,
3) acquiring sensor data indicative for measurement values of a physical parameter with respect to the patient's body using one or more sensing devices of the electronic device; and
4) providing, via the one or more communication interfaces of the control unit of the electronic device, the sensor data.

Optionally, the steps 1 to 3 are performed in the order above. Alternatively, the steps are performed in a different order.

The method may use the electronic device according to any aspect or embodiment described herein. Regarding the advantages of this method, reference is made to the description of the electronic device above.

The method may further comprise the following steps:
a) receiving, via one or more communication interfaces of the monitoring device, the authentication data;
b) determining, by means of an authentication module of the monitoring device, the validity of the received authentication data; and
c) recording, based on the determined validity of the received authentication data, the sensor data received from the electronic device.

Optionally, the steps a to c are performed in the order above. Alternatively, the steps are performed in a different order. For example, step a is performed after step 1 and/or step c is performed after step 3.

According to one embodiment, the steps 2; 3 and 4 are omitted if in response to step 1 the authentication fails.

The method may further comprise the following steps:
d) receiving, via one or more communication interfaces of the monitoring device, the expiry data;
e) determining by means of the monitoring device, whether or not the electronic device is beyond its expiry date;
f) give a warning if the expiry date is reached.

An electronic device according to a first embodiment is suitable for use in a medical operation, for examples general anaesthesia. For example, the electronic device comprises multiple electrodes as sensing devices for measuring EEG signals with respect to the patient's body. Furthermore the electronic device comprises a control unit, e.g. a processor is adapted to provide via one or more communication interfaces of the control unit, authentication data for authenticating the electronic device at a monitoring device. The authentication data comprises at least validity/invalidity information or data concerning sensor type, manufacturer and/or expiry data. In other words, the electronic device acts as an EEG sensor with extra functionalities.

The electronic device acquires sensor data indicative for measurement values of the physical parameter (EEG) with respect to the patient's body using the one or more electrodes; and provide, via the one or more communication interfaces, the sensor data, wherein the control unit comprises a memory for storing data, the memory stores expiry data to indicate and/or calculate an expiry date of the electronic device. The control unit is adapted to provide the expiry data via the one or more communication interfaces.

An electronic device according to the aforementioned embodiment further comprises a secure element adapted to provide at least a part of the authentication data. The secure element comprises for example a cryptographic key and/or a cryptographic algorithm. More preferably a cryptographic key is used on both sides, the monitoring device and the electronic device for authentication.

An electronic device according any of the aforementioned embodiments, wherein the authentication data is further indicative for the type of electronic device and/or for the type of a suitable monitoring device. For example, the EEG sensor is suitable for use with a specific anaesthesia monitoring device and/or for use with general EEG monitoring devices.

An electronic device according to any of the aforementioned embodiments or a combination thereof, wherein the control unit is configured to store sensor data and/or anaesthesia data and/or patient data in the memory.

An electronic device according to any of the aforementioned embodiments or a combination thereof, wherein the electronic device contains a flexible strip, containing a flexible circuit, with three or more EEG electrodes arranged thereon to collect EEG signals. Furthermore, the secure element is contained in the control unit, which is also attached, for example glued, to the flexible strip.

An electronic device according to the aforementioned embodiment, wherein the flexible strip is suitable to be arranged along the forehead to one temple of the patient. The fixed positions of the electrodes along the strip ensure proper placement of the sensing elements on the patient's forehead and temple. Preferably, one electrode is placed on the middle of the forehead of a patient, one on the temple of the patient and another one in between the forehead and the temple.

For example, electronic device communicatively connects the sensing devices and the control unit with the monitoring device by means of a cable.

A system for processing patient sensor data according to a first embodiment comprises the electronic device according to one of the aforementioned embodiments, and a monitoring device for monitoring the state of anaesthesia. The monitoring device comprises one or more communication interfaces and an authentication module. The system being adapted to receive, via the one or more communication interface of the monitoring device, the authentication data from the electronic device (EEG sensor). The monitoring device is adapted to determine by means of the authentication module the validity of the received authentication data, and record based on the determined validity of the received authentication data, the sensor data received from the electronic device. In other words, the sensor shall provide a physiological parameter with respect to the patient's body to the monitoring device. The physiological parameter for example EEG data shall be processed after the monitoring device has checked the sensor authentication with regard to sensor type, manufacturer and/or expiration data (e.g. expiry date, time of use). This guarantees the quality of the data by ensuring that the sensor is not used beyond its lifetime and/or shelf-life.

A system for processing patient sensor data according to the first embodiment, comprising a display. The display may be part of the monitoring device itself or connected to the monitoring device. The display may visualize data of the monitoring device and/or allow input of a user. The visualized data may among other be data received from the electronic device and/or data calculated using the received data from the electronic device. For example, received EEG data are used to calculate depth of anaesthesia of a patient and the display visualizes the state of anaesthesia of a patient based on the EEG data received from the electronic device.

Furthermore, information with regard to a remaining lifetime of the electronic device may be displayed.

A system for processing patient sensor data according to the first embodiment, wherein the monitoring device is adapted to disable the recording of sensor data if the received authentication data is determined to be invalid. In case authentication fails due to invalid authentication data, the monitoring system may generate an alarm signal for a user together with an invalidity information to replace the electronic device.

For example, in case authentication fails due to invalid sensor type or manufacturer data, the display freezes.

For example, in case authentication fails due to invalid expiry data, the system generates an acoustic and/or visual signal. The visual signal may for example be a message on the display, informing the user to replace the electronic device.

A system for processing patient sensor data according to one of the aforementioned embodiments, wherein once the sensor has been validated for monitoring, the monitoring device shall calculate from the physiological signal the effect of the first anaesthetic and/or second anaesthetic and determine an advisory action with regards to the infusion of the first, the second or both anaesthetics, when appropriate. Additionally, the sensor may carry physiological data of the patient allowing that the monitoring of this patient is resumed when connected to another valid device, for example another anaesthesia monitoring device or a general EEG monitoring device. For instance, a patient transferred from an operation room (OR) to an intensive care unit (ICU) or vice versa anaesthesia monitoring can be resumed in each ward.

In addition to or in combination with any aforementioned embodiments, the electronic device may also be configured to provide a signal depending on the type of procedure (medical operation respectively) it is expected to be used in and the kind of monitoring device it shall be connectable to, e.g., if it is solely for monitoring of data, if it is a target-controlled infusion device (TCI), if it is an intravenous anaesthesia infusion system, if it is an advisory depth of anaesthesia monitor or if it is a device embodying any combination of the aforementioned. When connected to one of the aforementioned monitoring devices or any combination, the monitoring device may be configured to send a signal to the electronic device requesting information with regard to the type of procedure it is expected to be used in, enabling the functions the electronic device is made for and freezing the rest. The electronic device is adapted to record this information into its memory to make this data available for the analysing device (particularly the monitoring device).

According to one aspect, the monitoring device is able to identify whether the electronic device is connected to solely monitor data, is a target-controlled infusion device (TCI), is an intravenous anaesthesia infusion system, is an advisory depth of anaesthesia monitor or is a device embodying any combination of the aforementioned, or vice versa.

A method for processing patient sensor data according to a first embodiment, comprising the steps of: providing, via one or more communication interfaces of a control unit of an electronic device, authentication data for authenticating the electronic device at a monitoring device. Furthermore, providing expiry data via one or more communication interfaces of the control unit of the electronic device to the monitoring device.

Additionally, the method comprises the step of acquiring sensor data indicative for measurement values of a physical (physiological) parameter with respect to the patient's body using one or more sensing devices of the electronic device; and providing, via the one or more communication interfaces of the control unit of the electronic device, the sensor data. The acquired sensor data comprise for example EEG data measured by the electronic device.

The method according to one the aforementioned embodiment further comprising the steps of:
receiving, via one or more communication interfaces of the monitoring device, the authentication data;
determining, by means of an authentication module of the monitoring device, the validity of the received authentication data; and
receiving, via one or more communication interfaces of the monitoring device, the expiry data;
determining, by means of the monitoring device, whether the electronic device has expired or not:
   recording, based on the determined validity of the received authentication data, the sensor data received from the electronic device.

The method may further comprise the steps of:
providing a signal to a user to verify the receipt of the authentication data;
providing a signal to a user indicative of the validity of the authentication data.

In addition, the method for processing patient sensor may comprise the step of disabling the recording of sensor data if the received authentication data, in particular the received data indicative of sensor type and/or manufacture is determined to be invalid by the monitoring device. In case authentication fails due to invalid authentication data the monitoring system may generate an alarm signal for a user together with an invalidity information to replace the electronic device.

Furthermore, in case authentication fails due to invalid sensor type or manufacturer data the display may freezes, disabling any further action.

In addition, the method for processing patient sensor may comprise the step, generating an acoustic and/or visual signal to indicate the authentication failed to invalid expiry data. For example, displaying a message on the display of the monitoring device, informing the user to replace the electronic device.

In other words, if the authentication is successful by means of the sensor type or manufacturer data and the electronic device is not expired the physiological parameter shall be recorded and monitored.

Furthermore, the method may comprise the step of displaying information with regard to a remaining lifetime of the electronic device. This information may for example be displayed on a display of the monitoring device. The remaining lifetime may help a user to decide whether the electronic device is still suitable for a planned use case. For example, an anaesthesia state of a patient is planned to last three hours and a remaining lifetime of the electronic device is four hours might lead to the decision to use a different electronic device, to minimize the risk to run out of lifetime.

In case the remaining lifetime is less than a certain predetermined lifetime the system may give this information to a user via a display of the monitoring unit. The information may be linked with a query to confirm that the electronic device should be used.

The method may use the system according to any aspect or embodiment described herein. Regarding the advantages of this method, reference is made to the description of the system above.

The idea underlying the invention shall subsequently be described in more detail by referring to the embodiments shown in the figures. Herein:

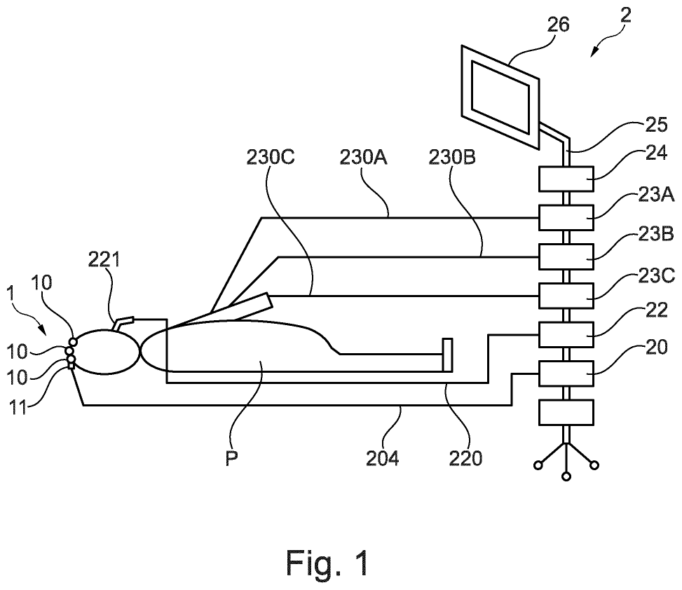

FIG. 1 shows a schematic view of a system for processing patient sensor data with an electronic device and for performing a target-controlled infusion;

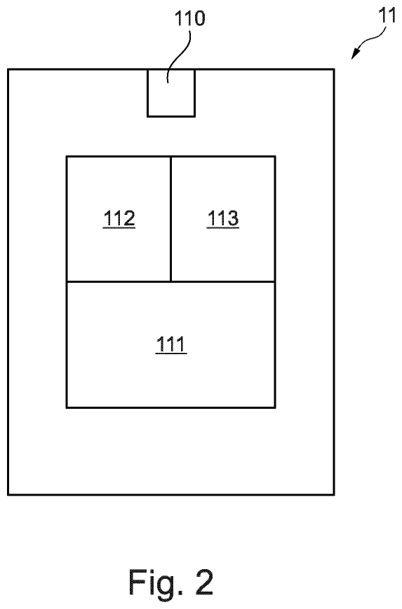

FIG. 2 shows a schematic diagram of a control unit of the electronic device of FIG. 1.

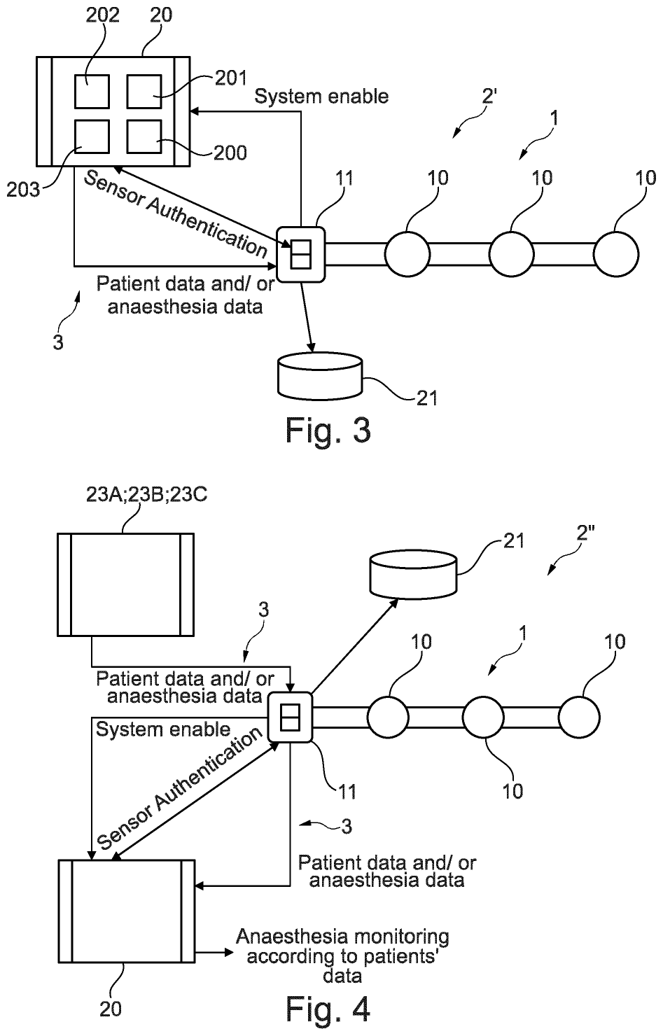

FIG. 3 shows a functional diagram of a system for processing patient sensor data with the electronic device of FIG. 1:

FIG. 4 shows a functional diagram of a system for processing patient sensor data with the electronic device of FIG. 1 and with an infusion device; and

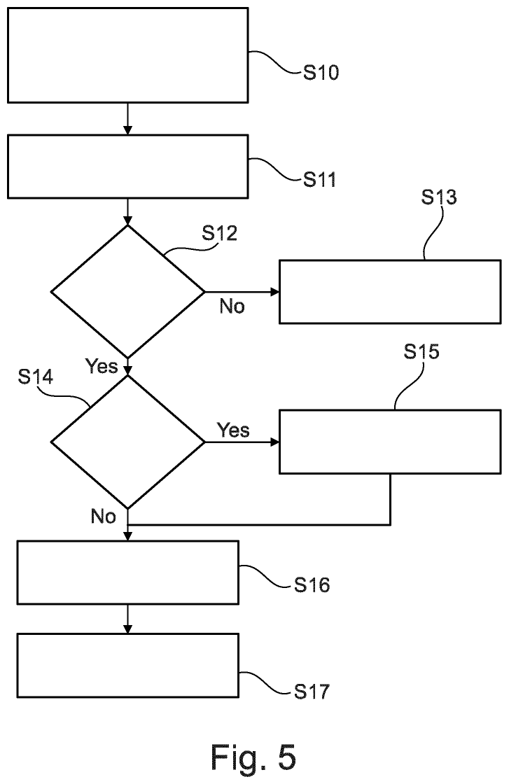

FIG. 5 shows a flow diagram of a method for processing patient sensor data with the electronic device of FIG. 1;

Subsequently, a device, system and method for processing patient sensor data, e.g., during an anaesthetic procedure, shall be described in certain embodiments. The embodiments described herein shall not be construed as limiting for the scope of the invention.

Like reference numerals are used throughout the figures as appropriate.

FIG. 1 shows a schematic drawing of a system 2 that may be used for example in an anaesthesia procedure for administering anaesthetic drugs, such as analgesic agents or hypnotic agents, e.g. propofol and/or remifentanil, to a patient P. In this setup multiple devices are arranged on a rack 25 and are connected via different lines and/or cables to the patient P.

In particular, infusion devices 23A, 23B, 23C such as infusion pumps, in particular syringe pumps or volumetric pumps, are connected to the patient P and serve to intravenously inject, via lines 230A, 230B, 230C, different drugs such as propofol, remifentanil and/or a muscle relaxant drug to the patient P in order to achieve a desired anaesthetic effect. The lines 230A-230C are for example connected to a single port providing access to the venous system of the patient P such that via the lines 230A-230C the respective drugs can be injected into the patient's venous system.

The rack 25 furthermore may hold a ventilation device 22 for providing an artificial respiration to the patient P, e.g., while the patient P is under anaesthesia. The ventilation device 22 is connected via a line 220 to a mouthpiece 221 such that it is in connection with the respiratory system of the patient P.

The rack 25 also holds a monitoring device 20 for monitoring the patient P. e.g., a bio-signal monitor, for example an EEG monitor which is connected via a wired and/or wireless connection, e.g., as shown in FIG. 1 by a cable 204, to an electronic device 1. As will be described in further detail below, electronic device 1 comprises a plurality of sensing devices 10, which in this example are attached to the patient P. The sensing devices 10 are configured and arranged for measuring a physical parameter with respect to the patient's P body, e.g., relating to a drug concentration within the patient P, for example by measuring a drug concentration in a patient's P breath or in the patient's P plasma (blood) compartment, or by measuring (electro-) physiological data, e.g., biological signals such as EEG or ECG signals, or by deriving indices such as the so-called bi-spectral (BIS) index.

In the present example, each of the sensing devices 10 is an electrode. The electronic device 1 is configured to measure electrical voltages (alternatively or additionally electrical currents) as physical parameter with respect to the patient's P body by means of the electrodes. In the example shown in FIG. 1, the sensing devices 10 are attached (and configured to be attached) to the patient's P forehead for monitoring the patient's P brain activity, e.g., during an anaesthesia procedure.

Sensor data acquired by means of the electronic device 1 are provided to the monitoring device 20, to the infusion device(s) 23A-23C and/or a control device 24. If necessary, a signal, for example an acoustic or optical signal is given to a user to adjust the control operation of the infusion device(s) 23A-23C to achieve a desired anaesthetic effect. Alternatively, the control device 24 adjust(s) the control operation of the infusion device(s) 23A-23C, corresponding to the sensor data acquired by means of the electronic device and outputs modified control signals to the infusion devices 23A-23C to achieve a desired anaesthetic effect. The acquired sensor data are indicative for measurement values of the physical parameter with respect to the patient's body.

The electronic device 1 of the example of FIG. 1 is an (electro-) physiological signal sensor. The electronic device 1 of FIG. 1 may also be referred to as a smart sensor. The electronic device 1 further comprises a control unit 11 which will be described in detail below.

The control unit 11 may be directly integrated into one of the sensing devices 10. Alternatively, the control unit may be a separate part comprised in the electronic device.

In addition, a control device 24 is held by the rack 25 which serves to control the infusion operation of one or multiple of the infusion devices 23A-23C such that infusion devices 23A-23C inject drugs to the patient P in a controlled fashion to obtain a desired effect, e.g. an anaesthetic effect. Control device 24 may, alternatively or in addition, control operation of ventilation device 22 and/or monitoring device 20 and/or other devices.

A display 26 is also mounted on the rack 25. The display 26 may visualize data of and/or allow input of a user to the various devices mounted on the rack 25, e.g., the monitoring device 20.

It is to be noted herein that the control device 24 may also be incorporated into the monitoring device 20, the ventilation device 22 or an infusion device 23A-23C, such that the control device 24 may be implemented by the respective device. It is further to be noted that the system 2 does not necessarily comprise ventilation device 22, infusion devices 23A-23C, control device 24, rack 25 and/or display 26. The individual devices of the system 2 are in communicative connection with one another via a wired or wireless connection.

Alternatively, or in addition, electronic device 1 may be configured, e.g., for measuring the concentration of one or multiple drugs for example in the breath of the patient P or to measure information relating to and allowing to determine, e.g., a bi-spectral index. It may embody, for example, a so called IMS monitor for measuring a drug concentration in the patient's breath by means of the so-called Ion Mobility Spectrometry. Other sensor technologies may also be used.

FIG. 2 shows the control unit 11 of the electronic device 1 of FIG. 1 in more detail. The control unit 11 comprises a communication interface 110, a secure element (SE) 111, memory 112 and a processor 113.

The communication interface 110 is adapted to establish a communication channel 3 (see FIG. 3) with the monitoring device 20 to exchange data between the electronic device 1 and the monitoring device 20. In general, the electronic device 1 may comprise one or more communication interfaces 110. Hence, the control unit 11 is adapted to enable a communicative connection between the monitoring device 20 and the electronic device 1.

The secure element 111 is adapted to provide at least a part of the authentication data. It has access to the memory 112 and/or comprises an internal memory storing a cryptographic key and a cryptographic algorithm (as, e.g., an encryption algorithm). The authentication data provided by the secure element 111 comprises the encrypted cryptographic key. Further, the authentication data comprises data indicating the type of the electronic device, e.g., the manufacturer, the sensing-device type and/or a type of a suitable monitoring device.

The memory 112 stores data. The memory 112 is at least in part non-volatile but may also comprise a volatile part. The control unit 11 is adapted to store sensor data acquired using the sensing devices 10, anaesthesia data and patient P data in the memory 112. Further, the memory 112 stores expiry data to indicate or for calculating an expiry date of the electronic device 1. In the present example, the expiry data includes the manufacture date of the electronic device 1 and a usage time. Alternatively, or in addition, the expiry data can comprise a specific expiry date. In general, the expiry data allows the monitoring device 20 to determine whether the electronic device 1 has expired and needs a replacement or can be used. The control unit 11 is adapted to provide the expiry data via the communication interface 110. Further, the memory 112 can store usage data of prior uses of the electronic device 1, e.g., indicating that the electronic device 1 has already been used in general and/or indicating the type of a procedure in which the electronic device 1 has already been used. The control unit 11 is adapted to provide the usage data via the communication interface 110. The electronic device 1 is adapted to store sensor data from the sensing devices 10 in the memory 112. Alternatively, no sensor data is stored at the electronic device 1. Further, the control unit 11 is adapted to receive data via the one or more communication interfaces 110, and to store the received data in the memory 112. Thus, the electronic device 1 may receive data from the monitoring device 20 and store it in the memory.

The electronic device 1 also comprises a processor 113. The processor 113 is adapted to execute instructions stored in the memory 112 to perform various steps of the electronic device 1 described herein, particularly:
providing authentication data via one or more communication 110 interfaces of the control unit 11 of the electronic device to the monitoring device 20 for authenticating the electronic device 1 at the monitoring device 20;
providing expiry data via one or more communication interfaces 110 of the control unit 11 of the electronic device 1 to the monitoring device 20;
acquiring sensor data indicative for measurement values of the physical parameter with respect to the patient's P body using the one or more sensing devices 10; and
providing the sensor data via one or more communication interfaces 110 of the control unit 11 of the electronic device 1 to the monitoring device 20.

The processor 113 is communicatively coupled to the memory 112. Further, the processor 113 is communicatively coupled to the secure element 111. Alternatively, the memory 112 and/or the processor 113 are included in the secure element 111.

The electronic device 1 enables sensor data recording by the monitoring device 20, e.g., embodied as an intravenous anaesthesia infusion system and/or an advisory depth of anaesthesia monitor, as well as to verify the authenticity of the electronic device 1, the time of use and/or expiry date. Moreover, the electronic device 1 may be configured to record sensor data and/or anaesthesia data and/or patient's P data. In an advisory (open loop) system at certain points of time, in particular prior to administering a drug dosage to a patient, a user interaction is required in order to manually confirm the operation.

FIG. 3 shows a system 2′ for processing patient sensor data. The system 2′ comprises the electronic device 1 and the monitoring device 20 of FIG. 1.

The sensing devices 10 (three in this example) of the electronic device 1 are communicatively connected to the control unit 11, in the present example by means of wires, so as to enable the control unit 11 to acquire sensor data from the sensing devices 10.

Further, the control unit 11 is communicatively connected with the monitoring device 20 by means of a communication channel 3 via the cable 204 (alternatively or in addition via another wired or wireless connection).

The monitoring device 20 comprises a communication interface 200, an authentication module 201, a memory 202 and a processor 203.

The communication interface 200 is adapted to establish the communication channel 3 with the communication interface 110 of the electronic device 1. In general, the monitoring device 20 may comprise one or more communication interfaces 200.

The authentication module 201 is adapted to receive and examine the authentication data provided by the electronic device 1. The authentication module 201 may be embodied in hardware and/or software.

The memory 202 is adapted to store data. The memory 202 is at least in part non-volatile but may also comprise a volatile part. If the authentication module 201 is embodied in software, the memory 202 may store the authentication module 201. The memory 202 may further be adapted to store sensor data from the electronic device 1.

The processor 203 is adapted to execute instructions stored in the memory 202 to perform various steps of the monitoring device 20 described herein, particularly:
receiving the authentication data from the electronic device 1;
determining, by means of the authentication module 201, the validity of the received authentication data; and
recording and/or effecting to record, based on the determined validity of the received authentication data, the sensor data received from the electronic device 1
receiving expiry data from the electronic device 1; and
determine, by suitable means, whether the electronic device 1 has expired or not.

The sensor data from the electronic device 1 may be stored in the memory 202 of the monitoring device 20 and/or in a storage memory 21. The storage memory 21 is communicatively connected with the electronic device 1 and/or with the monitoring device 20. The storage memory 21 may be arranged in the electronic device 1, in the monitoring device 20, in a dedicated storage system, e.g., mounted on the rack 25, in another system described with reference to FIG. 1, e.g., the control device 24, or located remotely.

When the monitoring device 20 is switched on and/or when the electronic device 1 is connected to the monitoring device 20, the authentication data is provided to the monitoring device 20. For example, the monitoring device 20 requests the authentication data or the electronic device 1 supplies the authentication data automatically upon a connection establishment.

The monitoring device 20 verifies that the manufacturer of the electronic device 1 is authorized for use with the monitoring device 20 and/or a selected procedure. Further, the monitoring device 20 verifies that the encrypted key is authentic, e.g., corresponds to the indicated manufacturer. Stated more generally, the authentication information can comprise an unencrypted first piece of information and an encrypted second piece of information. The first and second pieces of information correspond to one another. For example, the first and second pieces of information are identical and/or each comprise an indication of the same origin. The authentication module 201 is adapted to decrypt the encrypted key (encrypted second piece of information). For this purpose, the monitoring device 20 stores and/or receives a cryptographic key. This cryptographic key can be specific for a particular origin (e.g., manufacturer) of the electronic device 1. The monitoring device 20 may store a plurality of cryptographic keys. For example, the monitoring device 20 selects one of the plurality of cryptographic keys based on the first piece of information (e.g., an indication of the manufacturer). The decrypted cryptographic key (the decrypted second piece of information) is checked by the authentication module 201, e.g., compared with a key (piece of information) stored in the monitoring device 20 or accessible to the monitoring device 20.

Upon verification of the authentication data, the electronic device 1 is successfully authenticated. Then, the monitoring device 20 checks, by suitable means of the monitoring device 20, for example a processing unit, whether the electronic device 1 has expired or not. In this example, the monitoring device 20 checks the expiry data, whether the electronic device 1 beyond its recommended expiry date (shelf life) and/or has been already used for another procedure, or not. For this purpose, the electronic device 1 provides the expiry data and the usage data described above to the monitoring device 20. If the monitoring device 20 has not been able to successfully authenticate the electronic device 1 or if the electronic device 1 is determined to have been already used in another procedure than a procedure currently selected, the monitoring device 20 aborts the monitoring. If the electronic device is determined to have expired, the monitoring device 20 shows a (visible and/or audible) warning message to the user but proceeds with monitoring sensor data from the electronic device 1, alternatively the monitoring device 20 aborts the monitoring if the expiry date or the enhanced expiry date is reached.

Thus, after the authentication and the check of the expiry and a prior use, the operation of the system 2′ may be enabled. This may be triggered by a corresponding message provided from the electronic device 1 to the monitoring device 20 and/or vice versa.

In response to enabling the operation of the system 2′, the monitoring device 20 transmits patient data and/or anaesthesia data to the electronic device 1. The electronic device 1 receives the patient data and/or anaesthesia data and stores it, e.g., in the memory 112. Alternatively, or in addition, the system 2′ is configured such that in response to enabling the operation of the system 2′, the electronic device 1 transmits patient data and/or anaesthesia data to the monitoring device 20, wherein the monitoring device 20 receives the patient data and/or anaesthesia data and stores it, e.g., in the memory 202. The latter may apply when the electronic device 1 is used again for the same patient P.

Also, in response to enabling the operation of the system 2′ (or as an alternative thereto), the control unit 11 of the electronic device 1 records sensor data. The system is adapted to store the sensor data in the memory 202 of the monitoring device 20 and/or in the storage memory 21 and/or in the memory 112 of the electronic device 1.

Turning now to FIG. 4, a system 2″ for processing patient sensor data will be described. The system 2″ comprises the electronic device 1 and the monitoring device 20 of FIGS. 1 and 3, as well as at least one of the infusion devices 23A-23C of FIG. 1.

The infusion device(s) 23A-23C is/are communicatively connected with the electronic device 1 via a communication channel 3, for example, by means of a wireless or wired connection which may be a direct connection between the infusion device(s) 23A-23C is/are and the electronic device 1, or a connection via the monitoring device 20.

The authentication mechanism of the system 2″ is the same as described with reference to FIG. 3. According to FIG. 4, the system 2″ is configured such that (e.g., in response to enabling the operation of the system 2″), the infusion device(s) 23A-23C transmit(s) patient data and/or anaesthesia data to the electronic device 1, wherein the electronic device 1 receives the patient data and/or anaesthesia data and stores it, e.g., in the memory 112. The anaesthesia data may comprise data indicating a type of anaesthesia procedure, one or more parameter(s) of the anaesthesia, an algorithm etc. Alternatively, or in addition to storing the data, the electronic device 1 may be configured to transmit the patient data and/or anaesthesia data to the monitoring device 20 (e.g., in response to enabling the operation of the system 2″).

For example, when the electronic device 1 and/or the monitoring device 20 is/are connected to one or more intravenous anaesthesia infusion devices and/or target-controlled infusion (TCI) devices (e.g., infusion devices 23A-23C of FIG. 1) and only if the electronic device 1 is authenticated, by the time the patient data is introduced into the monitoring device 20 (or into another device communicatively connected therewith), a packet of data is transmitted from the monitoring device 20 to the electronic device 1. The electronic device 1 then stores this packet of data in the memory 112 of the electronic device 1. This memory 112 can be read by (e.g., its contents supplied to) the monitoring device 20 to potentially choose (by means of the monitoring device 20) the most adequate monitoring algorithmics (of a plurality of monitoring algorithmics) depending on the nature of the patient and the type of procedure it is being carried out. The monitoring device 20 also writes a case identifier in the electronic device 1 (e.g., transmits the case identifier to the electronic device 1 which stores it in the memory 112).

Optionally, the system 2″ is configured such that a data packet is sent from the infusion device(s) 23A-23C (e.g., intravenous anaesthesia infusion device(s) and/or target-controlled infusion (TCI) device(s)) to the electronic device 1 to record an indicator of a loss of consciousness and/or a recovery of consciousness, e.g., in the memory 112 of the electronic device 1. After concluding a procedure, e.g., an anaesthesia procedure, if the already used electronic device 1 is connected to the monitoring device 20 (or another suitable analysing and/or monitoring device), the authentication succeeds and the case identifier is known by the monitoring device 20. Thus, the case can be reviewed in the monitoring device with the information stored in the electronic device 1.

It will be appreciated that the electronic device 1, systems 2; 2″; 2″ and method described herein may be used to obtain (electro-) physiological data with the purpose of monitoring up to four of the main components related to depth of anaesthesia and sedation processes, hypnosis, analgesia, muscle relaxation and patients' movement. The general anaesthesia target is to provide an overall insensitivity and unconsciousness during the whole procedure. The basic elements of general anaesthesia include: unconsciousness (hypnosis), amnesia, analgesia, muscle relaxation, diminished motor response to noxious stimuli (patients' movement), reversibility. The electronic device 1 may be adapted to measure parameters indicative for one or more of these. The system 2; 2; 2″ may comprise multiple electronic devices 1 measuring different physical parameter with respect to the patient's P body (such as electrical voltages, electrical currents, breathing frequencies, etc.).

When using biomedical sensors (e.g., the electronic device 1) to acquire (electro-) physiological signals for recording and analysing it is advantageous to make patient P information available for the analysing device (e.g., the monitoring device 20). Paediatric and geriatric general anaesthesia could significantly differ from adults in the brain's activity response. Therefore, it may be provided that the electronic device 1 is configured to provide a signal to enable potentially different types of monitoring algorithmics depending on the different characteristics of the patient such as age, health condition, etc. as patient data.

The circumstances depending on the environment where the electronic device 1 is used can vary significantly. Therefore, the electronic device 1 may also be configured to provide a signal depending on the type of procedure it is being used for, e.g., sedation for ICU, surgical procedure or others. When an intravenous anaesthesia infusion device or a target controlled infusion device (TCI) is present (e.g., the monitoring device 20 embodied as such), the device may be configured to send a signal to the electronic device 1 requesting information with regard to the type of procedure being performed, e.g., a sedation or a surgical procedure (or an undefined procedure if it is not defined by the user). The electronic device 1 is adapted to record this information into its memory 112 to make this data available for the analysing device (particularly the monitoring device 20).

The electronic device 1 is able to identify whether the monitoring device 20 is connected to solely monitor data, is a target-controlled infusion device (TCI), is an intravenous anaesthesia infusion system, is an advisory depth of anaesthesia monitor or is a device embodying any combination of the aforementioned.

FIG. 5 shows a method for processing patient P sensor data.

The method starts at step S10. Therein the monitoring device 20 described above (which may be incorporated into an infusion device 23A-23C) is connected to the electronic device 1 described above. For example, the monitoring device 20 is an intravenous anaesthesia infusion system and/or an advisory depth of infusion monitor. The electronic device 1 may be referred to as smart sensor.

In a subsequent step S11 an authentication of the electronic device 1 is initiated. This comprises providing, via the one or more communication interfaces 110 of the control unit 11 of the electronic device 1, authentication data for authenticating the electronic device 1 at the monitoring device 20, and receiving, via the one or more communication interfaces 200 of the monitoring device 20, the authentication data.

In a subsequent step S12 it is determined whether or not the electronic device 1 is authenticated successfully. This comprises determining, by means of the authentication module 201 of the monitoring device 20, the validity of the received authentication data.

If the authentication was not successful, the method proceeds to step S13. Therein, no sensor data from the electronic device 1 is recorded by the monitoring device 20. For example, the system 2; 2′; 2″ is disabled.

If the authentication was successful, the method proceeds to step S14. Therein, it is determined whether or not the electronic device 1 is expired, based on expiry data, e.g., stored in the electronic device 1. This may be performed as described in more detail above. If it has expired, the method proceeds to step S15, wherein a warning is shown or otherwise signalled to the user, and proceeds further to step S16. Otherwise, the method directly proceeds to step S16. Optionally, it is also determined whether or not the electronic device has already been in use for another monitoring process based on usage data, e.g., stored in the electronic device 1. If this is the case, the method proceeds to step S13, otherwise it continues as illustrated in FIG. 5.

In step S16, the monitoring device 20 is enabled. This may include communicating, from the monitoring device 20 to the electronic device 1, that recording of sensor data shall be initiated.

In a subsequent step S17, sensor data and/or anaesthesia data and/or patient's data is recorded. This may include providing, via the one or more communication interfaces 110 of the control unit 11 of the electronic device 1, the sensor data and recording the sensor data received from the electronic device 1, e.g., by the monitoring device 20 and/or into the storage memory.

The idea of the invention is not limited to the embodiments described above, but may be implemented in a different fashion.

### LIST OF REFERENCE NUMERALS

1 Electronic device
10 Sensing device
11 Control unit
110 Communication interface
111 Secure element
112 Memory
113 Processor
2; 2′; 2″ System
20 Monitoring device
200 Communication interface
201 Authentication module
202 Memory
203 Processor
204 Cable
21 Storage memory
22 Ventilation device
220 Line
221 Mouthpiece
23A-23C Infusion devices
230A-230C Line
24 Control device
25 Rack
26 Display
3 Communication channel
P Patient

1. An electronic device for use in a medical operation, comprising:
one or more sensing devices for measuring a physical parameter with respect to the patient's (P) body; and
a control unit adapted to: provide, via one or more communication interfaces of the control unit, authentication data for authenticating the electronic device at a monitoring device; acquire sensor data indicative for measurement values of the physical parameter with respect to the patient's (P) body using the one or more sensing devices; and provide, via the one or more communication interfaces, the sensor data, wherein the control unit comprises a memory for storing data, the memory stores expiry data to indicate and/or calculate an expiry date of the electronic device, wherein the control unit is adapted to provide the expiry data via the one or more communication interfaces.

2. The electronic device according to claim 1, wherein the control unit comprises a secure element adapted to provide at least a part of the authentication data.

3. The electronic device according to claim 2, wherein the secure element comprises a cryptographic key and/or a cryptographic algorithm.

4. The electronic device according to claim 1, wherein the authentication data comprises an encrypted cryptographic key.

5. The system according to claim 1, wherein the authentication data is indicative for the type of the electronic device and/or for the type of a suitable monitoring device.

6. The electronic device according to claim 1, wherein characterized in that the control unit is configured to store sensor data and/or anaesthesia data and/or patient (P) data in the memory.

7. The electronic device according to claim 1, wherein the control unit is adapted to receive data via the one or more communication interfaces, and to store the received data in the memory.

8. The electronic device according to claim 1, wherein each of the one or more sensing devices is or comprises an electrode to acquire (electro-) physiological data of the patient (P) as sensor data.

9. A system for processing patient (P) sensor data, comprising:
the electronic device according to claim 1; and
a monitoring device, comprising one or more communication interfaces and an authentication module, the system being adapted to: receive, via the one or more communication interfaces of the monitoring device, the authentication data from the electronic device; determine, by means of the authentication module of the monitoring device, the validity of the received authentication data; and record, based on the determined validity of the received authentication data, the sensor data received from the electronic device.

10. The system according to claim 9, wherein the monitoring device is adapted to disable the recording of sensor data if the received authentication data is determined to be invalid.

11. The system according to claim 9, wherein an infusion device, wherein the system is adapted to monitor a depth of anaesthesia based on the sensor data.

12. A method for processing patient (P) sensor data, comprising:
providing, via one or more communication interfaces of a control unit of an electronic device, authentication data for authenticating the electronic device at a monitoring device;
providing expiry data via one or more communication interfaces of the control unit of the electronic device to the monitoring device; acquiring sensor data indicative for measurement values of a physical parameter with respect to the patient's (P) body using one or more sensing devices of the electronic device; and
providing, via the one or more communication interfaces of the control unit of the electronic device, the sensor data.

13. The method according to claim 12, comprising the steps of:
receiving, via one or more communication interfaces of the monitoring device, the authentication data;
determining, by means of an authentication module of the monitoring device, the validity of the received authentication data;
receiving, via one or more communication interfaces of the monitoring device, the expiry data; and
determining, by means of the monitoring device, whether the electronic device has expired or not; recording, based on the determined validity of the received authentication data, the sensor data received from the electronic device.
